(19) United States
(12) Patent Application Publication
(10) Pub. No.: US 2026/0091213 A1
(43) Pub. Date: Apr. 2, 2026

(54) SKIN PREPARATION DEVICE

(71) Applicant: NDM TECHNOLOGIES LIMITED, Loughborough, Leicestershire (GB)

(72) Inventor: Dewan Fazlul Hoque CHOWDHURY, Leicestershire (GB)

(21) Appl. No.: 19/112,239

(22) PCT Filed: Sep. 1, 2023
(86) PCT No.: PCT/GB2023/052271

§ 371 Date: Mar. 14, 2025

(30) Foreign Application Priority Data
Sep. 15, 2022 (GB) 2213572.7

(51) Int. Cl. A61M 37/00; A61B 5/00; A61B 5/145

(52) U.S. Cl. CPC A61M 37/0015; A61B 5/14514; A61B 5/685; A61M 2037/0046; A61M 2037/0061

(57) ABSTRACT

A skin preparation device (7) for preparing a region of skin (8) ready for delivering a therapeutic or cosmetic substance into the body of a subject through the skin or for measuring the concentration of a substance that is extracted from the region of skin, the device comprising an array of microneedles arranged to form artificial pores in the skin. The length and sharpness of the microneedles is limited to minimise trauma to the skin to minimise an inflammatory response. A plurality of micro-protrusions (5) may extend from an apex (1) of each microneedle or the tip of each microneedle may be bent to form a single, lateral micro-protrusion (6).

### TECHNICAL FIELD

This invention relates to a device and method for preparing a region of skin and to enhancing the diffusion of a substance into a human or animal subject through the skin, or extracting and measuring the concentration of a substance present in a human or animal subject.

### TECHNICAL BACKGROUND

The delivery of therapeutic or cosmetic agents into a subject through the skin has widely documented benefits. Similarly the detection and/or quantification of endogenous or exogenous substances is key to making diagnosis of medical conditions and non-medical conditions, e.g., cosmetic conditions of the skin. The substance may be present in any part of the body from where a representative sample may be removed to a platform where it is to be measured. This may involve the removal of the sample from the body of the subject. For example, in the case of measurement of a substance from the skin the substance may be extracted from the interstitial fluid using techniques including using a needle to prick or cause micro-ablation or abrasion of regions of the skin surface, focused sonic energy, microwave radiation, laser or light energy, and iontophoretic methods; this substance may then be measured externally to the body. The subject may be human or animal.

### SUMMARY OF THE INVENTION

The inventor has discovered that there is a relationship between trauma to the skin and the ability of a substance to diffuse through pores created in the skin. In particular, it was found that trauma on the skin causes an inflammatory response which leads to the movement of inflammatory mediators in the skin which, in turn, prevents increased diffusion of substance through the pores. Moreover, the repair to damaged skin that occurs also inhibits the movement of substance through the skin pores. This is explained in more detail below with reference to the figures.

It is therefore a key object of the invention to reduce or minimise the trauma caused to the skin when preparing the skin for extracting and measuring the concentration of a substance.

The invention teaches a device that forms superficial ablation and/or abrasion leading to regions appearing like pores on the surface of the skin using an array of microneedles which are designed specifically to ensure minimal trauma to the skin and thereby to prevent an inflammatory response that would lead to impeding the diffusion of substances into or out of said pores. It is understood to one skilled in the art that abrasion of the skin surface, whether mild or moderate, and the creation of pores in the skin will lead to some form of injury response by the skin, which is usually a response to repair the damage to the skin, and this is often described as an inflammatory response. This response can be further described as the aggregation of platelets and infiltration of leukocytes to the site of injury. Epithelialisation and newly formed granulation tissue, consisting of endothelial cells, macrophages and fibroblasts, act to cover the injured area to restore tissue integrity. It is a combination of these actions that leads to impeded diffusion (e.g. the outflow of glucose and other substances) from or into the skin.

It follows that inflammation in the context of this application is specifically described as the reaction of the skin to injury to the extent that it substantially impedes the further diffusion of substances from exiting or entering the skin. As such, “skin preparation” (or “skin prep”) in the context of the invention is the step of preparing the skin prior to the withdrawal of a substance from the skin or prior to the absorption of a substance (such as a drug) into the skin, wherein the preparing is to enhance diffusion through the skin (either exiting or entering) over prolonged periods of time. In the example of substances exiting the skin, this may be glucose that is present in the interstitial fluid.

It is acknowledged that inflammation will not be entirely prevented. It will occur to some extent irrespective of the extent of injury to the skin and the invention describes that this is the case but teaches that there is a balance between the extent of inflammation that leads to impeded diffusion of substances into or out of the skin, and inflammation that is mild in nature such that the skin does not fully recover nor lead to the ingress of protective chemicals and substances to the extent that sufficient substance(s) may diffuse out of or into the skin. In the example of substances exiting the skin, the substance (e.g. glucose) is able to exit through the surface of the skin in a quantity that can be measured using one or more types of sensor. Such a sensor may be one which uses wave forms (mid- and near infra-red, Raman spectroscopy, ultrasound, or radio-waves for example) or via direct measurement using an enzymatic reaction, for example reaction with glucose oxidase in the case of glucose measurements.

The inventor has established that quantities of glucose as low as picomolar concentrations can be detected using glucose oxidase enzyme based sensors. Optimal performance is seen where the method of measurement of the sensor is one that leads to the depletion of the substance being measured as part of the measurement process by virtue of the diffusion gradient between the inside of the skin and the surface of the skin that is created, thus acting to ensure a near constant diffusional pathway. In the event the sensor does not deplete the substance being measured at the outer surface of the skin, algorithms may be used to deduce the gradual reduction in diffusion of the substance out of the skin resulting from a build-up of the substance outside the skin, thus correcting for the reduced glucose concentration, by taking a cumulative approach to substance measurement.

Furthermore, superficial ablation/perturbation of the skin is described herein in further detail, in a manner that is both counterintuitive and contrary to the design of microneedle arrays that are widely described in literature. This is described further below. The inventor has established a means by which diffusion of substances can be elicited through the skin to the surface of the skin and in a manner that leads to minimal injury to the skin such that the skin's natural injury response does not impede the continuous diffusion of substances out of the skin for a period of time that is useful for continuous measurement of the concentration of the substance outside the skin, which can be correlated back to the concentration of the substance in the interstitial fluid and thus in the blood capillaries.

More particularly, the invention provides a skin preparation device as defined in claim 1, a substance measurement kit as defined in claim 16 and a method of preparing a region of skin as defined in claims 20 and 21. Preferred but non-essential features of the invention are defined in the dependent claims.

Microneedles according to the prior art are typically conical, tapering from a base to a sharp tip. As will be described below, microneedles according to the invention are more complex, comprising one or more micro-protrusions from the apex of each microneedle. When a distinction needs to be made, the term “apex” refers to the end of the main body of the microneedle that is furthest from the base, and the term “tip” refers to the sharpest extremity of a microneedle or micro-protrusion.

Any directional terms such as “up”, “down”, “top” or “bottom” refer merely to the orientation of the device shown in the drawings. It will be understood that, in use, the orientation of the device will depend on the region of skin to which it is applied and that the device may be manufactured, transported stored and sold in any orientation, while still falling within the scope of the claimed invention.

### THE DRAWINGS

FIG. 1a shows a glucose profile over the course of approximately 12 hours using a finger prick (top graph) v sensor data following a skin preparation method not according to the invention [method 1] (bottom graph). The vertical scales of the graphs use arbitrary units derived from the raw data in each measurement method so the absolute values are not directly comparable but the shape of the graphs can validly be compared. In practical applications of a transdermal measurement device, the directly measured value is converted to a glucose reading by calibrating against a finger prick value.

For the bottom graph, a device was used to gather the continuous data by application of the sensor to the skin in the manner described in the commercial product website sugarBEAT.com, whereby an enzymatic reaction takes place at 5 minute intervals and the raw data is sent by low energy Bluetooth® to a smart device, such as a mobile phone application, where an algorithm converts the raw data into glucose readings once at least one such reading is calibrated using a corresponding blood glucose value taken using a finger prick (or a predicted glucose value may also be used, i.e. one that is algorithmically derived). The finger prick data was taken hourly in this case and the device data taken at 5 minute intervals. The results show the low correlation between the two systems on the same person.

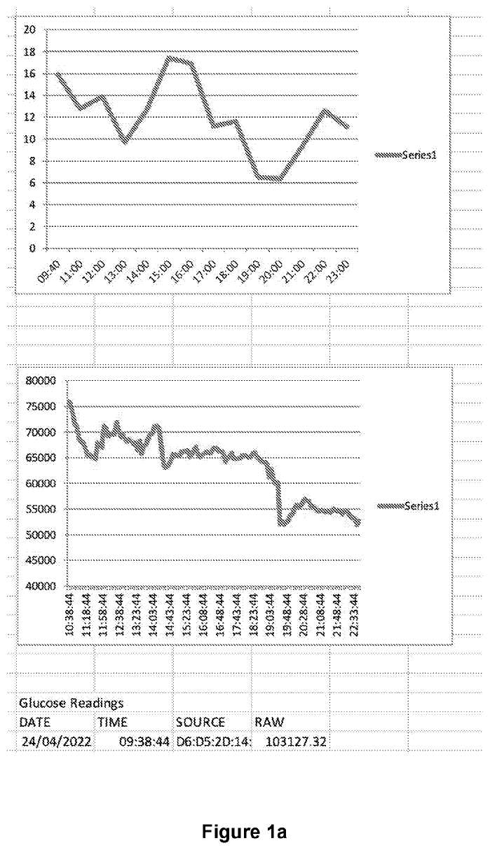

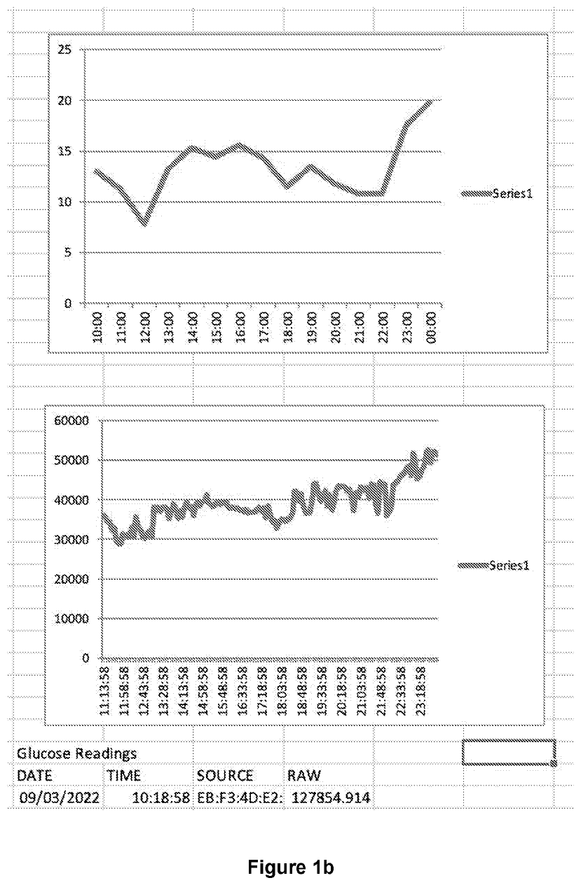

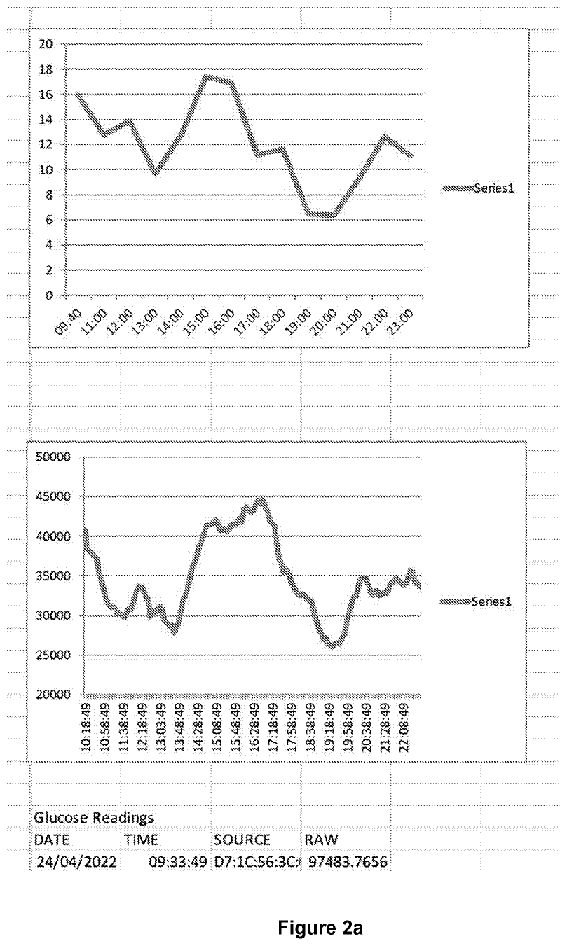

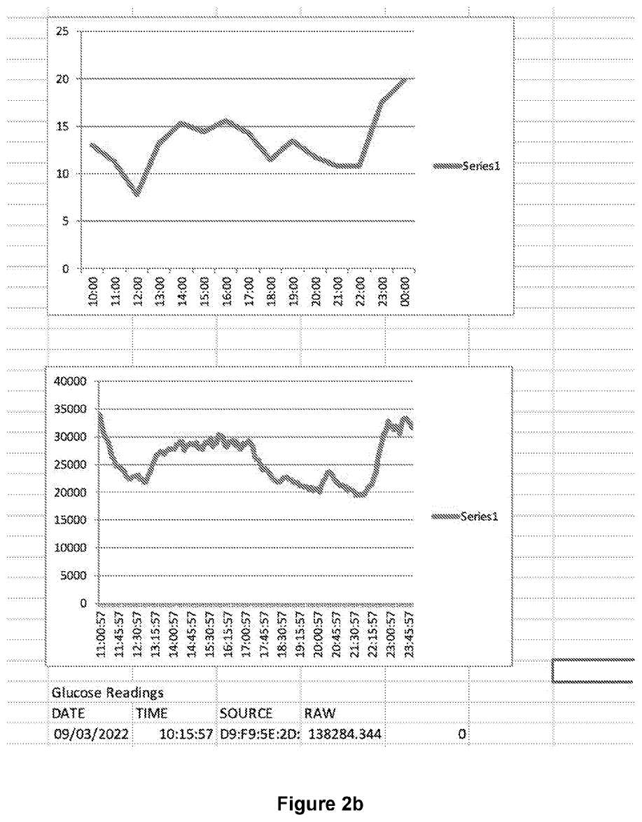

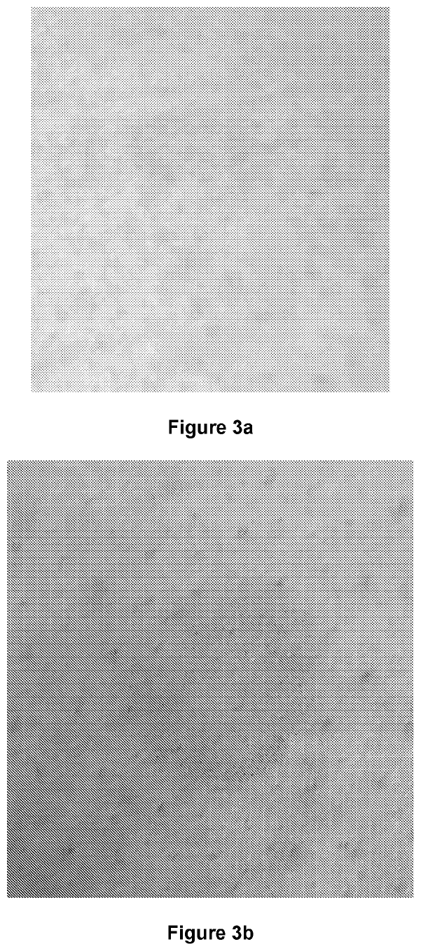

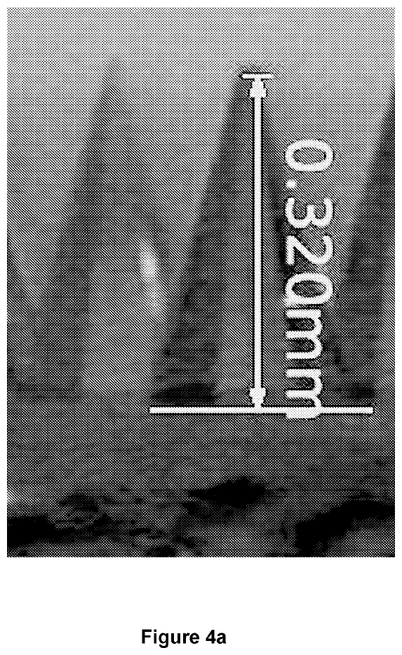

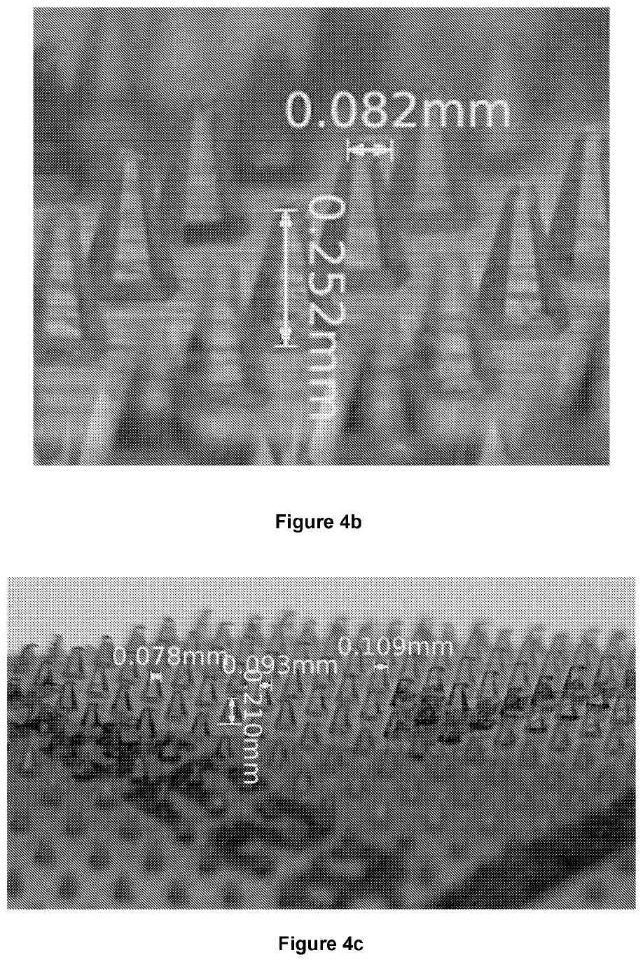

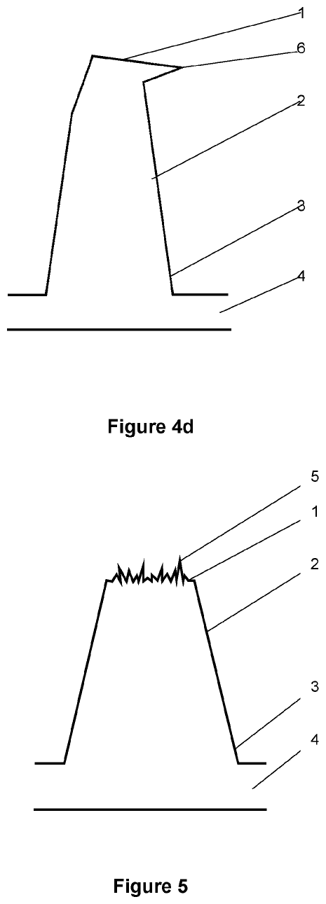

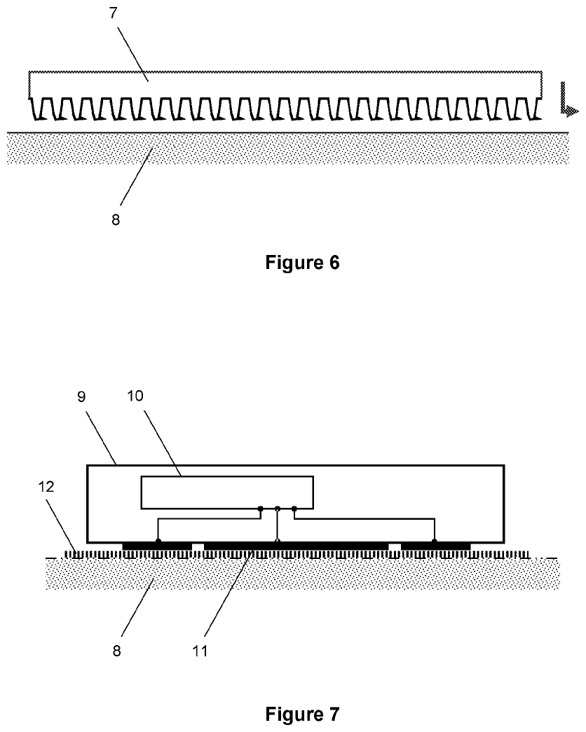

FIG. 1b is a repeat of the study of FIG. 1a, simply performed on a different day (as indicated by the time stamp). The results again confirm the low correlation between the two systems where the skin prep method is according to method 1 (contrary to the method of the present invention).

FIG. 2a is a second sensor that was worn by the same person on the same day as shown in FIG. 1a so the upper graph is the same as in FIG. 1a. However the skin prep method [Method 2] in this case is using the device and method according to the invention. The finger prick data is taken hourly and is shown in the first (top) graph, and the second (bottom) graph is the skin sensor data which is taken at 5 minute intervals. There is a clear and obvious correlation between these two systems where the skin prep device and method of the invention [method 2] is used. Note that, although the lower graphs in FIGS. 1a and 2a were from the same subject during the same time period, they were obtained using different sensors on different areas of the skin so their absolute values are not directly comparable but the shapes of the graphs can validly be compared. In practical applications, the values measured by the sensor would be converted to glucose readings by calibrating against a finger prick value.

FIG. 2b is a second sensor that was worn by the same person on the same day as shown in FIG. 1b, however the skin prep method [Method 2] is in this case using the device and method according to the invention. The finger prick data is taken hourly and is shown in the first (top) graph, and the second (bottom) graph is the skin sensor data taken at 5 minute intervals. There is a clear and obvious correlation between these two systems where the skin prep device and method of the invention [method 2] is used.

FIG. 3a shows an area of skin having been treated by the device and method of the invention (i.e. via method 2) where redness, and therefore any inflammatory response, is absent; the photo being taken at the end of 14 hours' wear of the device.

FIG. 3b shows an area of skin having been treated by a device and method not of the invention (i.e. via method 1) where redness, and therefore inflammation, is shown to be present even after the 14 hours' of wear of the sensor.

FIG. 4a shows an example of a microneedle patch not according to the invention (e.g. as used in method 1).

FIG. 4b shows an example of a microneedle patch according to the invention as used in method 2.

FIG. 4c further illustrates an example of a microneedle patch according to the invention as used in method 2, whereby the perpendicular arrangement of the sharp microneedle tips is clearer.

FIG. 4d shows a schematic of a single needle from the microneedle patch of FIG. 4c.

FIG. 5 shows a schematic of a single needle from a microneedle patch according to an alternative embodiment of the invention.

FIG. 6 schematically shows how a device according to the invention may be used to prepare a region of the skin.

FIG. 7 schematically shows the use of a sensor to extract a sample from a prepared region of skin.

It has been noted in literature that skin permeation has been enhanced through the use of microneedles, for the enhanced absorption of drugs and cosmetic substances through the skin. A wide range of needle configurations and geometries have been noted. Additionally, it has been noted in particular by Prausnitz et. Al., (Georgia Institute of Technology) that microneedles can be inserted in the skin to create pores, deep enough to then allow the removal of interstitial fluid (often by applying a vacuum to the skin) to allow substances present in the analyte to then be measured using sensors situated outside the skin. In particular the state of the art has been focused on the insertion of sharp microneedles into the skin to draw out substances from interstitial fluid under the stratum corneum layer of the skin. The inventor has discovered that there is a strict relationship between needle tip sharpness and the ability to meaningfully remove analyte from the skin for the purpose of measuring the analyte on the surface of the skin. FIG. 1a shows the glucose profile over the course of approximately 12 hours using skin preparation method 1 and FIG. 2a indicates the glucose profile for the same person simultaneously measured using a second identical device and sensor but using skin preparation method 2. It is clear from skin preparation method 2 that there is consistent enhanced diffusion of the analyte through the newly created pores in the skin, with peaks and troughs responding to food/glucose intake, whereas in the device where method 1 of skin preparation was used there is minimal diffusion through the skin and a downward trend/signal drift without any response is seen. A further example of this is shown in FIGS. 1b and 2b respectively.

Furthermore, FIG. 3b shows that method 1 causes distinct redness or erythema on the skin, which is the skin's reaction to injury. FIG. 3a shows that there is no erythema or any other effect on the skin arising from the use of method 2.

The core difference between the two methods is that in method 1 the tip sharpness of the needles was less than 30 μm (in terms of the width of the tip of the needle), and the needle height was greater than 300 μm. It is clear that a combination of the needle height and tip sharpness has led to sufficient trauma on the skin and below the skin surface such that the skin has reacted. It is postulated that the skin reaction is an inflammatory response and such an inflammatory response leads to the movement of inflammatory mediators in the skin-prepped region which, in turn, prevents increased diffusion of analyte through the pores (as seen in FIGS. 1a and 1b), and erythema/redness of the skin albeit transient. Moreover, the repair to damaged skin which occurs also inhibits the movement of glucose through the skin pores.

In contrast, the method and device of the invention does not elicit a local inflammatory response such that increased and enhanced diffusion is seen (as shown in FIGS. 2a and 2b). In method 2 the tip (or apex) of each microneedle was at least 40 μm in diameter and up to 140 μm in diameter, and the microneedle height was between 150 and 1000 μm. It was found that this needle tip sharpness leads to the least amount of skin trauma and most consistent levels of diffusion through the skin where the tip sharpness (as indicated by the horizontal measurements shown in FIGS. 4b and 4c) is between about 60 and 120 μm, and more preferably between 80 and 120 μm. In the event that the needle apex is not circular, the diameter should be measured as the minimum dimension in the plane perpendicular to the axis of the main body of the microneedle.

It was further established to be beneficial if the apex comprises a rough surface and not a smooth flat surface, the surface roughness being defined here as a surface with one or more micro-protrusions on the surface of several μm, and up to 20 μm in any one dimension. The surface of the needle apex is more akin to a fine grain sandpaper than to a smooth flat surface, as shown schematically in FIG. 5. There may be a large number (say, at least ten) of the micro-protrusions 5, each protruding from the microneedle apex 1 by less than a width of the microneedle apex, to cause the apex of the microneedle to be rough. These surface protrusions 5 may all be parallel to the axis of the microneedle body but preferably they each protrude at an angle from the apex 1. Different micro-protrusions 5 may be at multiple different angles leading to random surface roughness, though the degree of roughness could also be engineered in the manufacture process to create uniform angled particles or protrusions on the surface. For example, if cuboid particles were adhered to the flat surface apex, either mechanically bonded using ultrasound or chemically bonded using a liquid adhesive agent such as an acrylic adhesive, these adhered particles would create uniform surface roughness. Alternatively such surface roughness could be created by creating a mould tool where the needle is fabricated by embossing or micro-imprinting, whereby the inner surface of the mould tool has angled surface features designed within it such that the moulded part contains an imprint of these features.

Furthermore it was established that a superficial pore of greater surface area with minimal skin injury resulted from an arrangement whereby the microneedle apex in the original configuration was prepared as a sharp tip, i.e., having a tip diameter of between 1 μm and 25 μm approximately, but then the tip 6 was forced into an angle approximately perpendicular to the longitudinal axis of the main body/column 2 of the needle to form a bent microneedle with a lateral micro-protrusion 6, as shown in FIGS. 4c and 4d. The bent tip may protrude from the main body of the microneedle by between 1% and 100% of a base diameter of the microneedle, and more preferably by between 10% and 30% of the base diameter. This counterintuitive design had the impact of producing highly consistent prolonged diffusion of analyte from below the skin to the surface of the skin, without a degree of injury to the skin that caused an inflammatory response that could have impeded the diffusion of substances out of the skin.

FIG. 4d is a 2-dimensional side view depicting flat needle apex 1, needle column/main body 2, needle base 3 and associated substrate 4. At a micro level, this arrangement has a greater surface roughening effect than a sharp needle tip would have and, because of the lateral micro-protrusion 6, when the microneedles are pressed into the skin there is slight lateral movement of the apex of the microneedles in the order of micrometres. It follows that this movement, combined with a microneedle tip that is now perpendicular to the vertical column of the microneedle, acts to abrade/pick on the surface of the skin thus enhancing the efficiency of ‘skin-prep’ in terms of the period over which such extraction or absorption could occur, compared with where sharp tipped microneedles (i.e., tips less than 30 μm in diameter) are used. This methodology is both counterintuitive and contrary to that which is taught in the state of the art where vast resources have been deployed to created microneedles with super-sharp tips for skin penetration.

It was furthermore established that the needle height plays a role in that, even with a flat needle apex, it is possible to press the entire length of the microneedle into the skin and create a relatively large puncture in the skin. However, this is also a function of the force applied to the needle, noting that in that a bed of needles the applied force is distributed across the needles on the array, thus reducing the force on each individual needle. The array of microneedles according to the invention preferably has at least 50 microneedles per square centimetre surface area with a needle height of up to 1000 μm, and up to 150 μm microneedle apex diameter. In the event that excessive force is applied to the needles, they will pierce through the skin layers and lead to skin trauma, therefore the force applied should be sufficient to only cause the needle to perturb the outermost layer of the skin, the stratum corneum, as this is the layer that acts as a barrier to either the ingress of material into the skin or loss of moisture and salts out of the skin. The depth of penetration of the microneedle may be as much as 150 μm in order to achieve this level of skin perturbation, depending on the thickness of the stratum corneum on different subjects. Quantitatively speaking, the force applied is less than 200 newtons per square centimetre. Therefore, the force exerted per microneedle where there are at least 50 microneedles per square centimetre is less than 4 newtons. Preferably the force is in the range 30 to 180 newtons per square centimetre for an array of 50 microneedles distributed over 1 square centimetre with microneedle apex diameter of 70 to 120 μm and needle body diameter of less than 150 μm, and more preferably 40 to 160 newtons.

The microneedles themselves could be manufactured from a range of materials known in the state of the art including polymers such as polypropylene, methacrylates, polytetrafluoroethylene, and also metals and ceramics.

These are critical findings that none of the prior art has disclosed nor could have envisaged or predicted. In order to detect the differences between the two methods outlined above, the inventor required direct and specific access to skin sensors of a sensitivity of sub-nanomolar and picomolar (analyte) glucose concentrations, as used in this invention, and systematic approach to skin preparation to determine the optimum means of achieving skin preparation to allow glucose to diffuse in a constant manner through the newly created pores. The optimum results are achieved based on ensuring there is not such damage to the skin that leads to a local inflammatory response being elicited, which in itself is achieved using a microneedle array apex geometry that is counterintuitive. It follows, therefore, that the needle height, needle width, apex sharpness and force applied relative to the needle density are important attributes of a device making use of the invention, in addition to the needle geometry, and these parameters have not been previously disclosed in this context based on studies on human subjects.

Such microneedle arrays with sharp tips that are at an angle equal to or less than 90° to the vertical column of the needle may be created by producing microneedles with supersharp tips (made using an embossing technique for example) then applying even pressure across the surface of the microneedles with the tips placed against a substrate, thus causing the tips to collapse in one direction. The direction of collapse of the microneedle tips is engineered such that it is in the general direction of lateral movement of the microneedle apex when the microneedle array is pressed into the skin, thus enhancing the impact of the lateral needle tip. Preferably, the tips are pressed against a penetrable substrate such as rubber, such that the tips can bend but not break. Furthermore, pressing the tips against a penetrable substrate allows the tips to bend back on themselves, i.e. through an angle more than 90°. The tip that is flattened protrudes by at least 1% and up to 100% of the base diameter of the microneedle. It should be noted that microneedles having a similarly “bent” structure could be formed using a process such as moulding, which does not involve an actual bending step.

Accordingly, the invention includes forming pores using an array of microneedles which minimises trauma to the skin to prevent an inflammatory response. FIG. 6 schematically shows a patch or device 7 comprising an array of microneedles that may be applied to a region of the skin 8 by pressing it against the skin, accompanied by a slight lateral movement as indicated by an arrow. The array of microneedles does not penetrate the skin to a depth greater than 150 μm, and more preferably no greater than 100 μm, but preferably at least 50 μm, where the needle arrays are applied specifically to the forearm, upper arm, or upper chest, back and thigh (since these regions of the skin offer the lowest thickness of skin/epidermis and these regions are more prone therefore to inflammatory reactions as well as allowing the most optimal diffusion of glucose out of the skin under the correct conditions, i.e., where skin trauma is not elicited).

The term “pore” is used herein to define a passage that allows the transport of a substance from where it may be removed to be measured and its concentration determined or, conversely, the transport of a substance into the body. The pores may be created by physical projections with tip diameter, main body length and width as described herein.

Substances to be measured may include though are not limited to: glucose, lactic acid (for neonates in critical care, and for athletic performance training/monitoring), phenylalanine, prostaglandin, and drugs including valproate, phenytoin, lithium and blood alcohol.

Substances that may be introduced into the body via pores created using a method and device according to the invention include but are not limited to: vaccines, biological drug molecules such as peptides and proteins such as teriparatide, and small molecules such as zolmitriptan and fentanyl. The drug could be applied in a liquid or gel reservoir patch against the skin where the micro-needles have been used to porate or ‘prep’ the skin.

The microneedles used in the method of the invention will penetrate the skin sufficiently to enable interstitial fluid to flow to the surface of the skin or substances to diffuse from the surface into the interstitial fluid. The minimum depth will therefore be the thickness of the stratum corneum which is generally greater than 10 μm, depending on the age of the skin and its location on the body. A penetration depth of at least 50 μm is therefore preferred.

The poration of the skin removes the variability of the natural pores and follicles in the skin, and their behaviour in terms of pore closure or blockage due to physiological or external environmental conditions. Sufficient pores will therefore be created to ensure that, for a given surface area, the amount of interstitial fluid extracted exceeds—and preferably greatly exceeds—that which would be extracted through the naturally occurring pores. For example if 10 μg of a substance can be extracted passively from the natural skin or actively through forced perspiration for example, then the poration described in this invention should lead to the removal of sufficient substance (at least 15 μg in this example) such that the variability inherent in extracting passively or through forced perspiration, is diluted out and makes a negligible contribution to the measurement. In other words, the contribution of the extraction through natural pores should be small enough to be ignored as noise in the measurement signal, taking into account the accuracy of measurement that is deemed acceptable. The extraction efficiency is deemed standardized under these circumstances.

The artificially formed pores described in this invention may contract upon withdrawal of the needles, but to a minimal extent given that the primary function of the pore formation is the surface perturbation of the outer most layer of the skin, rather than deep pore formation into layers of the skin that elicit an injury response. The extent of contraction of the surface of the skin may therefore be expected to be in the region of up to 20% of the microneedle diameter. Therefore where an array of needles occupies (i.e., the needle diameter and tip directly make contact with) 5% of the area of the skin, gives rise to pores occupying approximately 3% of the area of the skin.

Various methods are known for determining the number and cross-sectional area of both natural and artificial pores in a region of skin. Video microscopy can be used to capture a 2D image of a sample of skin, or of a silicon replica taken from a region of skin and image analysis tools applied to make the required measurement. Alternatively laser-scanning microscopy can be used to capture the 3D morphology of a skin sample or replica.

FIG. 7 schematically shows the use of a sensor 9 to analyse a medium extracted from a region of skin 8 that has been prepared in accordance with the invention (and is therefore indicated schematically with a broken line). The sensor may comprise electrodes 11 facing the skin 8, which are in turn connected to a controller 10 in the sensor 9. A nylon membrane 12 may be used as a means to actively encourage the medium to flow through the pores formed in the skin. The nylon membrane 12 may have a plurality of pores with a pore size of greater than 0.2 μm. The nylon membrane absorbs the glucose as it arises to the surface of the skin. The pores in the membrane allow the glucose to travel rapidly and efficiently from the surface of the skin leading to a capillary pull/diffusion through the body of the membrane, instead of just accumulating at the interface between the skin and the membrane. Such a capillary diffusion or pull effect helps to maintain the diffusion gradient out of the skin, which is critical to ensure that capillary diffusion continues uniformly. The glucose that then reaches the sensor (e.g. an enzyme-based electrode), is subsequently depleted/used up during each sensing cycle. The enzyme complex contained in the electrode can be present on the surface of the electrode where the electron generated from the reaction with the glucose is picked up and translated as an amperometric signal.

It will be understood that the sensor may be any suitable sensor, such as an enzyme-based sensor or an optical fluorescence type sensor.

It will also be understood that the array of microneedles may be arranged on any suitable support. For example, the microneedles may be located on a patch, on a flat plate at the end of an insertion device, or on a roller. The microneedles may be pressed into the skin using the thumb or index finger, or they may be rolled onto the skin so that each row of microneedle is introduced to the skin one row at a time to avoid the bed of nail effect and control the exertion force on the skin. The microneedles may be introduced to the skin as part of a guiding frame that allows the needles to be in a fixed position.

1. A skin preparation device for preparing a region of skin to enhance subsequent diffusion of a substance out of or into the region of skin, the device comprising:
a microneedle array having a plurality of microneedles arranged to form artificial pores on the skin surface; wherein
each microneedle comprises a main body defining an axis from a base to an apex and
each microneedle comprises one or more micro-protrusions at the apex of the main body.

2. A skin preparation device according to claim 1, wherein each microneedle comprises a plurality of the micro-protrusions at the apex.

3. A skin preparation device according to claim 2, wherein each microneedle comprises at least ten of the micro-protrusions each protruding from the apex by less than a width of the apex.

4. A skin preparation device according to claim 2, wherein the apex of each microneedle comprises a surface and wherein the micro-protrusions protrude from the surface, an axis of each micro-protrusion being inclined relative to the surface.

5. A skin preparation device according to claim 4, wherein the micro-protrusions protrude from the surface at multiple different angles of inclination.

6. A skin preparation device according to claim 4, wherein the micro-protrusions all protrude from the surface at the same angle of inclination.

7. A skin preparation device according to claim 2 wherein the micro-protrusions protrude from the apex of the microneedle by up to 20 μm.

8. A skin preparation device according to claim 1, wherein each microneedle comprises a single micro-protrusion protruding from the apex of the main body an axis of the micro-protrusion being inclined relative to the axis of the main body.

9. A skin preparation device according to claim 8, wherein the axis of the micro-protrusion is inclined substantially at a right angle relative to the axis of the main body.

10. A skin preparation device according to claim 8, wherein the inclined micro-protrusion is the bent tip of an originally conical microneedle.

11. A skin preparation device according to claim 8, wherein the micro-protrusions of all the microneedles in the array are inclined in the same direction.

12. A skin preparation device according to claim 8, wherein the micro-protrusion protrudes from the main body of each microneedle by between 1% and 100% of a diameter of the base of the main body.

13. A skin preparation device according to claim 1, wherein the apex of each microneedles has a diameter of between 40 μm and 140 μm, preferably between 80 μm and 120 μm.

14. A skin preparation device according to claim 1 wherein each microneedle has a height of between 150 μm and 1000 μm.

15. A skin preparation device according to claim 1, wherein the tip of each micro-protrusion has a sharpness of between 1 μm and 30 μm.

16. A substance measurement kit comprising:
a skin preparation device comprising a microneedle array having a plurality of microneedles arranged to form artificial pores on the skin surface, wherein each microneedle comprises a main body defining an axis from a base to an apex, and each microneedle comprises one or more micro-protrusions at the apex of the main body; and
a sensor for detecting the concentration of a substance in a medium that flows out of the skin through pores created by the skin preparation device.

17. A substance measurement kit according to claim 16, further including an extractor operable to encourage the medium to flow out of the skin through the pores.

18. A substance measurement kit according to claim 17, wherein the extractor comprises a porous membrane.

19. A substance measurement kit according to claim 18, wherein the porous membrane is formed from nylon.

20. A method of preparing a region of skin to enhance subsequent diffusion of a substance out of or into the region of skin comprising the steps of:
a) applying a skin preparation device to the region of skin to form artificial pores in the skin, the device comprising: a microneedle array having a plurality of microneedles arranged to form artificial pores on the skin surface; wherein each microneedle comprises a main body defining an axis from a base to an apex; and each microneedle comprises one or more micro-protrusions at the apex of the main body, and
b) a step selected from a list comprising:
i) inserting into the skin the microneedles of the device to a depth no greater than 150 μm, and preferably no greater than 100 μm; and
ii) applying a lateral movement of the order of micrometers to the array of microneedles as the microneedles are inserted into the skin.

21. (canceled)

22. A method of preparing a region of skin as recited in claim 20, further including the step of determining a concentration of a substance in a subject including the step of using a sensor to detect the concentration of the substance in a medium that flows out of the tissue through the pores created by the skin preparation method.
